(19) United States
(12) Patent Application Publication
(10) Pub. No.: US 2012/0215157 A1
(43) Pub. Date: Aug. 23, 2012

(54) Device, Methods, and Control for Sonic Guidance of Molecules and Other Material Utilizing Time-Reversal Acoustics

(75) Inventors: James G. Berryman, Danville, CA (US); Raghu Raghavan, Baltimore, MD (US)

(73) Assignee: THE REGENTS OF THE UNIVERSITY OF CALIFORNIA, Oakland, CA (US)

(21) Appl. No.: 13/208,756

(22) Filed: Aug. 12, 2011

Related U.S. Application Data
(63) Continuation of application No. PCT/US2010/024486, filed on Feb. 17, 2010.
(60) Provisional application No. 61/153,555, filed on Feb. 18, 2009.

(51) Int. Cl. A61M 37/00; F17D 3/01

(52) U.S. Cl. 604/22; 137/13; 137/803

(57) ABSTRACT

The present invention provides for a method of controlling mass movement of fluid material within a field of interest comprising using time reversal acoustic focusing. The time reversal acoustic focusing can be is used for simultaneous spatial and temporal focusing of acoustic energy to control the duration of localization and/or direction of movement of material within tissue or liquid within tissue. Both delivery of material and persistence with respect to target locations can be enhanced by focusing of sonic waveforms or transmissions towards targeted areas in a field of interest, such as within a patient.

### RELATED PATENT APPLICATIONS

This application claims priority as a continuation application of PCT International Patent Application No. PCT/US2010/024486, filed Feb. 17, 2010, which claims priority to U.S. Provisional Patent Application Ser. No. 61/153,555, filed Feb. 18, 2009, which are hereby incorporated by reference in their entireties.

### STATEMENT OF GOVERNMENTAL SUPPORT

The invention described and claimed herein was made in part utilizing funds supplied by the U.S. Department of Energy under Contract No. DE-AC02-05CH11231. The government has certain rights in this invention.

### FIELD OF THE INVENTION

The invention relates to the field of guiding materials in porous and other media.

### BACKGROUND OF THE INVENTION

The current methods used for the retrieval of stuck oil and the remediation of hazardous waste fluids from underground involve shaking large volumes of the ground simultaneously. This approach requires very large amounts of seismic energy input into the ground.

### SUMMARY OF THE INVENTION

The present invention provides for a method for controlling mass movement of a fluid material within a field of interest comprising:
(a) transmitting at least one original signal of sound waves from at least one array of transceivers located outside of a field of interest, or from at least one transceiver located inside the field of interest;
(b) receiving the at least one original signal with at least one receiver or transceiver within the field of interest as a received signal; or receiving the at least one original signal in parallel with multiple transceivers located outside the field of interest if the said original signal is transmitted from within the field of interest;
(c) processing the at least one received signal by a processor using an acoustic time-reversal geometric mean interpolation method to generate a signal content relationship between the original signal and the at least one received signal;
(d) designing a modified acoustic waveform using the generated signal content relationship to produce a specific acoustic effect in the field of interest near the at least one receiver; and
(e) transmitting the modified acoustic waveform from the at least one array of transmitters outside of the field of interest to modify mass movement of materials within the field of interest.

The present invention also provides for a system for controlling the flow of material introduced into a region of interest comprising:
(a) an array of at least one repositionable array of at least three transmitters located outside of the region of interest;
(b) an output control for the at least three transmitters causing pulse trains to be transmitted from each transmitter in the array;
(c) at least one receiver receiving the pulse train within the region of interest; and
(d) a processor executing geometric mean interpolation software contained in the processor of a computational scheme that computes mass flow based on a transmitted waveform and known characteristics, comprising: at least some existing database of general characteristics within the region of interest or specific patient characteristics derived from radiological imaging within the region of interest to simulate a received wave train at a designated point in tissue in the region of interest, and the processor can iterate until a transmitted waveform results in a designed mass flow, which designed waveform would then be transmitted by the at least three transmitters.

In some embodiments of the invention, the system is an ultrasound system and the transmitters are ultrasound transmitters.

In some embodiments, the material is introduced into the region of interest by injection.

In some embodiments, the field of interest is tissue within a subject. In some embodiments, the subject is a multicellular living organisms, such as a mammal. In some embodiments, the mammal is a human.

The present invention can be used in conjunction with ultrasound imaging technology. The present invention can be used for controlling mass movement of delivered material within a subject, such as a human patient. This control effect can be accomplished by a methods comprising: (a) at least one original signal of sound waves is transmitted from at least one array of transmitters outside of a field of interest in the patient; (b) the at least one original signal is received by at least one receiver within the field of interest as a received signal; (c) the at least one original signal is processed by time-reversal acoustic procedures to generate a mathematical and physical causal relationship between the original signal and the received signal; (d) the generated causal relationship is used to design a modified original signal that will produce a specific intended acoustic effect in the field of interest near the at least one receiver by geometric mean interpolation; and (d) the modified original signal which is then transmitted from at least one of the at least one array of transmitters outside of the field of interest to modify mass movement of materials within the field of interest.

The present invention can be used for controlling flow of material injected into tissue. This can be accomplished by a method comprising: (a) providing a set of at least one ultrasound transmitter array(s) located outside of the region of interest in the tissue; (b) providing at least one signal from the at least one ultrasound transmitter, which can be either internal or external to the field of interest and within and/or without a patient, if used in a medical procedure; (c) providing at least one receiver/transmitter located within the tissue; and (d) implementing time-reversal acoustics by steps comprising (i) recording the at least one signal from the at least one ultrasound transmitter as a pulse or pulse trains transmitted from the at least one transmitter in the array, (ii) modifying the received waveform in a pre-determined manner to affect an acoustic result within the tissue including geometric mean interpolations, (iii) time-reversing the modified signal to form a time-reversed waveform (first signal in, last out and last signal in, first out), (iv) further modifying the time-reversed waveform including scaling, normalization, and amplification in a pre-determined manner to affect an acoustic result within the tissue, and (v) re-transmitting from the transmitter array as a modified waveform pulse.

The present invention also provides an ultrasound device or system for controlling the flow of material injected into a region of interest within tissue. The device or system can comprise: (a) an array of at least one repositionable array of one or multiple ultrasound transmitters located outside of the region of interest of the tissue, (b) output control for the transmitters causing pulse trains to be transmitted from the transmitters in the array, (c) at least one receiver for receiving the pulse train within the region of interest; and (d) a processor capable of executing software contained in the processor of a computational scheme of geometric mean interpolation that computes mass flow based on known characteristics of at least some of the skull and brain tissue, meninges and blood vessels, to simulate the received wave train at a designated point in tissue in the region of interest.

In some embodiments, it is advantageous for speed of processing that the receivers be transceivers, i.e., have transmission capability in addition to receiving capability. When feasible and safe in the applications, the receivers are transceivers.

The invention can be used for guiding materials in porous and other media. In some embodiments, the invention is used to guide therapeutic substances which are introduced into the body by the use of ultrasound applied from an array of one or more transducers to induce acoustic radiation force and streaming of the interstitial fluid in brain parenchyma. The beam is directed towards the target tissue to enhance fluid flow and transport the target material to the desired site more efficaciously and in a much shorter time than that normally occurring passively, or with pump-driven infusion alone, or due to endogenous bulk flow in the tissue. It will also concentrate flow more efficiently to the region of the desired treatment volume. Another important field of application of this invention is in distributing an infusate over the broad but thin area of cerebral cortex for possible treatment of neurological diseases such as Alzheimer's disease. The method can be additionally effective when the target tissue is imaged with acoustic, magnetic resonance (MR), or X-ray or computed tomographic (CT) imaging and related techniques. The method has further advantages when the flowing infusate can be imaged with acoustic, MR or CT techniques; such imaging can be enhanced by including small ultrasound, MR or CT contrast agents in the injected material.

The invention can also be used for mitigating fluid flow problems in the earth, i.e. underground. Such applications include, the retrieval of stuck oil and the remediation of hazardous waste fluids from underground. The method introduced in this invention is very targeted and localized, and permits much smaller amounts of seismic energy to be input to achieve the same ends.

The invention can also be used for data compression applications, both those related to the present applications such as drug perfusion or dislodging stuck underground oil or fluid contaminants and also to other applications to very large 2D and 3D seismic wave datasets used in the oil industry when searching for oil and gas underground.

The guidance and/or control of the flow of materials in porous media is referred to under the term “Acoustic Shepherding”.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows a schematic representation of a procedure on a skull with some particular embodiments of the invention.

FIG. 2 shows a representation of an array that has a hexagonally arranged array with 25 transducers in a cluster.

FIG. 3 shows a representation of an array that has a hexagonally arranged array with 37 transducers in a cluster.

FIG. 4 shows a series of image figures representing how the focal spots move as the value of a control parameter is changed in the interpolation scheme described below to obtain desired waveforms in a region between two receivers.

FIG. 5 shows an endovascular embodiment of the invention within the scope of the generic invention.

FIG. 6 shows an application of the invention in intrathecal delivery, where an objective is to enhance delivery of drug or therapeutic particles into cortical tissue.

FIG. 7 shows a schematic representation of problems faced when infusion of therapeutic solutions into the prostate gland is attempted.

FIG. 8 shows a schematic representation of one practice of the present invention on a prostate gland.

FIG. 9 shows a schematic representation of another practice of the present invention on a prostate gland.

FIG. 10 shows a flow chart of a specific process within the generic concepts according to the present invention.

FIG. 11 shows a flow chart of a specific process within the generic concepts according to the present invention.

FIG. 12 shows a process by which interpolation of response functions may be performed to allow Acoustic Shepherding at points intermediate between two or more receiver locations.

FIG. 13 shows different temporal protocols for the Acoustic Shepherding process.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a sensor” includes a plurality of such sensors, and so forth.

These and other objects, advantages, and features of the invention will become apparent to those persons skilled in the art upon reading the details of the invention as more fully described below.

The invention provides for a method for sending a signal to a remote location somewhere off a grid of available sensors. Sending such a signal could be for purposes of communicating or irradiating of a certain region with sound for dislodging or moving an object by using acoustic/ultrasound vibrations, or for establishing a pressure gradient to encourage a packet of fluid (or materials suspended or dispersed or dissolved therein) to flow in a particular direction. Embodiments of the present invention for these and other applications are described herein.

The present invention provides for a method comprising a time-reversal procedure that has been developed by the inventors using geometric mean interpolation to provide a predetermined sonic output for use in procedures. The time reversal procedure as described makes use of the reciprocity of the acoustic Green's function to permit refocusing on a known (or possibly unknown) source location by recording a wave train received at a set of transducers, time-reversing these signals using geometric mean interpolations (first in last out, last in first out) and then rebroadcasting them back into the acoustic medium. If the signals received and recorded were from a single source, the reciprocity property of the wave equation effectively guarantees that some part of the time-reversed signal will indeed reconverge on the original source location.

The present invention involves the use of time-reversal acoustics. The invention uses soundwave geometric mean interpolation. In some embodiments, modest amplitude acoustic focusing may be used to direct perfusion of drugs to desired locations within a body, and even lodged solids (even clots, thrombi or clot chips). In some embodiments, a seismic application might be to dislodge oil, other fluids, or contaminants that are stuck underground and that should be encouraged to flow towards a particular well-bore for retrieval and/or subsequent disposal. Thus, at least one concern addressed in such embodiments of the invention is a method to refocus sound or seismic energy back into a target medium at points other than the at least two source points. In geometric mean interpolation, a time reversed waveform is transmitted with geometric mean interpolation of amplitudes so that the modified waveform pulse is received by the tissue at a desired point intermediate between at least two receiver/transmitter locations.

It is necessary that there are at least two sound sources available. When used in a medical application, these might be placed within two or more catheters on opposite sides or in a surrounding perspective of a tumor, or a large region within which it is desired to provide a fairly uniform distribution of some drug or other fluid medication. These two sound sources can each be used to send a signal from their current locations to an external array in a location outside the body (such as on the surface of the body) of a subject. By using a short burst of sound (i.e., a ping), from each of these two transducers (geophones) in turn, and subsequently recording the resulting signals at the external array, benchmark empirical Green's functions are established for the organ and/or tissue(s).

It is necessary that there are at least two sound sources available. When used in a geophysical or seismic application, at least two well-spaced boreholes may be provided between which is a region where it is desired to create focused vibrations to dislodge a contaminant, or to encourage stuck oil or other fluids to flow. These two sound sources can each be used to send a signal from their current locations to an external array at the surface of the earth or in a location underground (such as in a borehole, mine or cave). By using a short burst of sound (i.e., a ping), from each of these two transducers (geophones) in turn, and subsequently recording the resulting signals at the external array, benchmark empirical Green's functions are established for the host earth medium.

The practice of the invention involves one or more of the two following features as described herein. Sound wave propagation and movement in liquid environments can be used to adjust duration and movement of mass within a liquid. That is, even though there may be significant and existing local forces (capillary action, Brownian motions, diffusion and other forces causing mass transfer within the liquid), proper application and focusing of sound waves in the liquid can alter movement of mass within the liquid by a controlled mechanism. The use of mathematical oversight concerning the spatial orientation of the wavefronts, and temporal arrangement as well as the physical properties of sound waves such as the magnitude or intensity using time-reversal acoustic techniques can provide enhanced focus and quality/quantity to the transmission of sound waves, which will significantly enhance the performance of directed mass movement and/or persistence of movement within a liquid environment.

The term “liquid environment” is not specifically limited to large open areas of liquid, such as within a patient. The term includes not only liquid volume within vessels and ducts, but also intercellular and intracellular liquids. The forces applied to the masses in the liquid environments can be used to reduce or enhance existing mass transfer patterns in specific (targeted) locations so that mass within that liquid environment will persist longer, distribute more rapidly, or distribute more efficiently (i.e., with less loss of the directed effusate) in a desired direction and speed than ambient conditions allow. Similarly, in seismic application, the seismically induced pressure gradient causes in the pores of the earth medium (rock) to flow via Darcy's law.

The inventions described herein include methods, procedures, systems, apparatus and processes for influencing mass movement within liquid environments, such as the movement of delivered materials within patients, such as catheter-delivered solids, liquids or gels delivered to or from vessels in a patient.

The present invention can be used in the following three applications. These applications, which are termed Acoustic Shepherding, are: (1) Mass Movement Control, (2) Directed Mass Movement, and (3) Mass Movement Prevention. In the process of Mass Movement Control, the focused ultrasound is used to contain delivered material within a region of the field of interest of a patient by inducing localized pressure gradients and barriers. An aspect of the exercise of this first related technology is that first a planned focusing transmission of ultrasound or sonic waves has been generated by the underlying process described and explained in greater detail herein. Then, transmission of the focused ultrasound (by designed amplitude, frequency, pulse shape, pulse duration, intensity, directionality and other vectors defining the transmission), is used to restrain movement of delivered mass within the region of interest for the patient. This focused sound may be used essentially to retain a delivered mass (e.g., bolus, dose, dispersion, concentration or volume of material) approximately at a desired location. For example, if an active agent has been delivered into the putamen, adjacent to the white matter tracts of the internal capsule, enhanced liquid flow in the white matter region would dissipate that drug from that location within a period of time X under normal flow and fluid movement circumstances with a time X, which could be quite fast and not allow significant further filling of the putamen. The dissipation would theoretically (without significant flow in that region) be a radial mass migration of the material away from a center of the mass and away from the putamen. By providing focused sound waves emitted from transmitters focused antiparallel to a number of channels of movement away from the center of the mass, timed pulses can restrain mass movement by using acoustic pressure pulses to push back against the normal lines of mass migration.

Mass Movement Control (focusing sound to produce directional flow) can be used to focus the propagation and absorption of transmitted ultrasound energy in a direction parallel towards a delivery end of a catheter, the ultrasound pushing delivered material distally, reducing backflow up the catheter. Backflow of this type occurs because the pressure from the flow pushes the tissue away from the catheter, allowing a tissue-free channel for fluid flow moving the material up the length of the catheter, and reducing the flow of fluid into the parenchyma itself. Therefore one aspect of the Mass Movement Control is to be able to direct ultrasound along the length of the catheter.

Directed Mass Movement involves designing focused ultrasound transmissions from at least one transmitter array outside of the field of interest to at least one receiver within the field of interest. Directed Mass Movement is related to Mass Movement Control in that the focused ultrasound or sonic transmission is directed at delivered material, but instead of specifically attempting to restrain normal movement, a planned movement of the mass is intended, with the sonic forces causing portions of the delivered mass to move in a planned direction. For example, drug delivery within the brain is sometimes limited to accessible delivery locations because veins, arteries and capillaries are present at only specific locations and are not necessarily uniformly distributed. An operator selects an available vessel that is most convenient and most functionally located (e.g., one would not want to deliver material into a vessel a few centimeters from a target cite if the movement of liquid within the vessel is directed further away from the site). Having chosen an appropriate delivery vessel, the drug is delivered into and out of that vessel to the delivery site, and the Directed Mass Movement is performed by focusing pulses of the ultrasound according to a planned program that will actually cause movement of the delivered material in an intended direction based upon use of the focused acoustics and the plan.

Mass Movement Prevention is closely related to the first two specific technologies described above, but has a different type of application. In this application, material (drugs, indicators, dyes, markers, etc.) are delivered into a region where there are multiple available pathways for movement, as within the prostate. When materials are normally delivered into the prostate, there is a greater tendency for them to be removed by ducts going into the prostatic urethra, rather than being distributed within the prostate tissue itself. The original transmission time reversal acoustic analysis is performed on the region and an imaging plot of the region is taken so that location and direction of the ducts is evaluated. A planned ultrasound transmission application is designed to increase lateral (relative to the direction of movement of the fluid within the ducts) mass movement pressure so that material movement into the prostatic urethra can be reduced by application of forces away from the ducts, towards the point of delivery of the material.

One process useful for the treatment of the prostate is to practice the time-reversal and modified waveform technology described herein so that pressure is directed from inside the prostate outwardly to reduce amounts of fluids entering a prostatic urethra.

The time-reversal acoustic (TRA) technique described herein can be used to achieve the result described in Choi et al. (“Non-Invasive, Transcranial and Localized Opening of the Blood-Brain Barrier using Focused Ultrasound in Mice,” Ultrasound in Med. & Biol., Choi, James J. et al., Vol. 33, No. 1, pp. 95-104, 2007; which is incorporated by reference). The present invention can be used to open the Blood-Brain Barrier by combining the TRA analysis and reprogramming of the transmission of ultrasound to be used with the ultrasound procedure.

The present invention provides for a method to control flow of material provided to or in (e.g., delivered, carried, transferred, naturally generated, decomposition products and/or injected into) tissue. The manner of delivery is not critical as long as its location of delivery can be determined and/or controlled.

The invention provides for a method comprising: (a) providing at least one ultrasound transmitter; (b) providing at least one phased signal from the at least one array of ultrasound transmitters, the signal being a sonic wave, sonic waveform or series of sonic waves/waveforms that have known acoustic properties (e.g., frequency, amplitude, duration, energy, etc.); (c) providing at least one receiver/transmitter (acoustic transducer) located within a tissue of a subject, and (d) implementing time-reversal acoustics, comprising (i) recording (or performing real time analysis without persistent recording, as with flash memory, field programmable gated array, or ASIC chip) at least one signal from the at least one ultrasound transmitter as a pulse or pulse trains transmitted from the at least one transmitter in the array, (ii) modifying the virtual received waveform in a pre-determined manner, such as to define a transmission protocol of phased array output that will have predicted or planned sonic activity in the vicinity of the receiver, (iii) time-reversing the modified waveform to develop information for a virtual time-reversed waveform, (iv) modifying the virtual time-reversed waveform, which may include scaling, normalization, and amplification, in a pre-determined manner to affect an acoustic result within the tissue and (v) re-transmitting from the transmitter array as a modified waveform pulse.

In some embodiments, the (a) providing step (such as that in paragraph [0051]) further comprises providing at least one array of ultrasound transmitters located outside of the region of interest in the tissue. In particular embodiments, the transmitters are positioned in the least invasive manner, such as outside the body. In some embodiments, when the tissue is brain tissue, the transmitters are in contact with or proximal to the skull or scalp in delivery of mass to the brain. An array is a phased array comprising at least three transmitters. In some embodiments, the phased array comprises at least 8, at least 16, at least 32, or at least 128 transmitters/receivers. In some embodiments, one transducer can be used either/both as receiver or transmitter, but phasing requires three or more transducers.

In some embodiments, the (c) providing step (such as that in paragraph [0051]) comprises at least one receiver/transmitter (acoustic transducer) located within the tissue, in receiving mode being capable of providing acoustic information that it receives to an operator of the system, including a memory device or processor, while in transmitting mode it can emit small amplitude pulses to be recorded by the external array(s) of transducers.

In the practice of this type of method in medicine, in a particular embodiment, the tissue is brain parenchyma, and the external transmitter array is arranged on the outside of a skull of a live patient, and at least one receiver is at one or a set of pre-determined positions along an axis of one or more catheters introduced into the brain parenchyma. The term array is used, even where there may be a single transmitter to cover situations where a single transmitter is moved outside the region of interest, the various locations are noted, and separate transmissions from the same transmitter at different locations are used to develop preliminary phased sonic wave data used in the time-reversal acoustic treatment of the data to design simultaneous sonic transmissions from multiple transducers, such as 2 or more transducers, that will provide local mass movement control as desired and planned.

The method as described herein may include intraparenchymal introduction of therapeutic material performed within 10 cm, within 5 cm, within 4 cm, within 3 cm, within 2 cm and within 1 cm or less (e.g., within 5, 4, 3, 3 or 1 mm) of the at least one receiver in a region of tissue of interest in the overall process. In some embodiments of the invention, the method as described herein may be practiced with a single transducer array on the outside of the skull, or multiple (2, 3, 4, 5, 6) transmitter arrays outside of the skull or other region of interest in the patient. In a particular embodiment, the tissue is that of an internal organ (such as the brain) of a live patient and the receivers/transmitters are inserted into the tissue of interest. There may be more than one receiver/transmitter within the tissue, as for example, along an axis of a catheter, or at the tip of more than one catheter or cannula, and a transmission protocol for at least one transmitter array may introduce a time sequence of decreasing pressure amplitudes at various receiver locations, to induce a net influence on the direction or vectors of flow in a vicinity of at least one of the more than one receivers.

It should be appreciated at this point that time reversal is an important quality control in the performance of the sonication or sound wave procedures of the technology described herein. When a sound wave is transmitted into an object, such as a patient, having an amorphous or having a diversity of composition, the transmission and progression of the sound wave through the patient is highly affected by the specific properties of the path through the patient. In medical or mass delivery procedures, this variability must be considered in treatment of individual patients to optimize the medical results intended. The time-reversal acoustics use the known information of the transmission, the received information of that transmission at a specific location in the patient (in the vicinity of the receiver), and time reverses the received information to determine the actual sonic effect at the receiver from the actual transmission, and calculates and measures the reduction or diminution of the signal, due to the wave spreading, scattering, and/or attenuation loss to heat, and the properties of the received signals.

In particular, the causal response becomes known between the transmitting and receiving transducers. This information (also known as the Green's function between the two points) is the information required for establishing the desired protocols for intraparenchymal therapy delivery design. Based on this information, the location, direction and properties of subsequent transmissions can be planned or designed to provide specific acoustic results at the target location to be able to control mass movement within the liquid environment. Planning can also include movement of the location and/or distribution of transmitters, frequency of pulses, amplitude of pulses, duration of pulses, and the like at the target site. Time reversal acoustics allows the practitioner of the invention to focus sound pressure without knowing the properties of the medium in detail. If the properties of the medium are not known to the practitioner, either by consulting existing databases, or by inference from other imaging methods such as magnetic resonance or computed tomographic imaging, then another approach to focusing would be by the technology known as “phased array technology”. This ability of time reversal acoustics to provide a focus within the medium can be successful because it is not necessary to know the medium properties to practice the invention.

In an embodiment of this invention, the desired focal point is optimized by a learning method for geometric mean interpolating weights of local and acoustic parameters. Contrast agents visible under ultrasound may be co-infused with the desired intraparenchymal therapy, or infused just prior to the therapeutic procedure. The Acoustic Shepherding described in this invention may thus be monitored in real time. Several properties of waveforms including their phases in the array, their amplitudes, and other features are amenable to adjustment by learning algorithms including statistical estimation and neural network methods. A multiplicity of transmitters may be used to enhance amplitude of the pulse at a specific receiver location when a time-reversed waveform is provided to at least some of several transmitters, and the signal had been recorded at the specific receiver location.

Multiple receivers may be positioned along at least one catheter(s) and the catheter(s) may be introduced into blood vessels. The effect of the transmitted ultrasound reversed waveform is focused, transmission of the focused transmitted waveform causing location specific alteration of blood brain barrier permeability so that drugs introduced into blood vessels is delivered into the central nervous system. The drug may be injected into a peripheral blood vessel and then it may enter into catheterized vessels by standard pharmacokinetic phenomena, and then controlled by the methods of the present technology. At least one algorithm in executed software may be used to optimize the focused power for safe and appropriate blood brain barrier alteration. The software may further include consideration of pharmacokinetic phenomena to ensure adequate dosing and residence of the drug or therapeutic molecule in CNS tissue.

A modified waveform pulse may be received within or adjacent to a volume of the tissue at a desired point intermediate between at least two receiver locations so that a delivered portion of drugs is retained within the volume of the tissue for a length of time longer than predictable for diffusion and perfusion factors in the volume without transmission of the modified waveform. This procedure is referred to as mass transfer stabilization, as opposed to mass transfer focus.

In a particular embodiment, the tissue targeted in these procedures is brain parenchyma, and the transmitter array comprises multiple transmitters arranged outside of the skull. The application of the invention has been generically described in delivering a specified waveform to an intraparenchymal location where an ultrasound receiver is, or has been, placed. It is an aspect of this invention that the array of transmitters is placed outside the skull, and it is a key component of this device that we take advantage of the time-reversal properties of the acoustic waves to deliver enough power to be efficacious within tissue without requiring significant power in the transmitter that could otherwise be damaging. It is equally an advantage of this invention that very low frequencies are not required for use. The usual, and profound, disadvantage of ultrasound in brain imaging has been the “acoustic opacity” of the skull (due to the acoustic impedance mismatch and resulting high reflectivity) which often requires both high power and low frequencies for adequate transmission into the parenchyma and subsequent detection through the skull again. The invention takes advantage of time-reversal in “focusing” a time sequence of acoustic power to a “point” in both space and time. (This of course requires the usual assumptions of time-reversal acoustics to work well, which includes the dominance or importance of the phase relationships of the signals that combine after scattering and reflection over any differences of sound wave speeds and or inertial densities. Also, the impedance mismatch between skull and brain will still be an impediment to high transmissibility, but having multiple arrays of transmitters should help to overcome this problem.) Thus the requirements of sufficient power in the acoustic signal is reduced since we add up the integrated power in the time domain (i.e., the total transmitted energy over the duration of the pulse train) to provide higher power at the intended receiving point or location. For the same reason, we do not need to use low frequencies which, although they transmit better through the skull, are also unfortunately more dangerous, being known to cause cavitation in tissue. Thus the time reversal method allows us to use lower power from individual transmitter arrays, a less dangerous range of frequencies, and last but not least, place the transmitters outside the parenchyma. Devices which have multiple higher amplitude acoustic transmitters within parenchyma are believed liable to result in more adverse events than transmitter arrays that remain outside. The internal receivers/transmitters are for one-time use, while the external transmitter arrays can be more expensively constructed and are meant for multiple re-use.

In another unique practice, the at least one receiver comprises encapsulated objects arranged at positions in blood vessels, the encapsulated objects being guided to the positions by magnetic stereotaxis; and then the ultrasound is focused by the time-reversal acoustics methodology for alteration of blood brain barrier permeability, causing drugs introduced into blood vessels to be delivered into central nervous system regions of the patient.

The methods herein may also be practiced where the tissue is brain parenchyma, the transmitter array is arranged on the outside of the skull, and the at least one receiver is an encapsulated object arranged at positions within brain tissue, the encapsulation having migration restraining physical elements of chemically active functionality to restrain migration of the receivers. Physical or chemical “tails” or migration impeding functionality may be added to the encapsulated object to reduce its migration, and the initial estimated focusing of ultrasound or other sonic transmissions used to gather original data may also control movement of the receivers. Long chemical tails such as polyoxyethylene, siloxane polymers, fluorinated polymers, polyamide polymer tails or other inert and biologically acceptable species may be used for the chemical and/or physical tails. Harmless soluble tails may be used with time dissolution of the tails and capsules after use, as with controlled dissolution polymers such as polyvinyl alcohol, polyoxyethylene polymers, polyvinylpyrrolidone, gelatin, polyamylose, and the like.

Additionally, enhancement of the movement of large particles such as viruses or cells may also be expected to be similarly controlled due to the acoustic radiation pressure on such particles themselves. This effect is expected to be negligible for small proteins, in which case the maximum benefit of the acoustic forces is on the fluid carrying the drug. Thus, the placement of more than one cannula which in any case can be called for by the pharmaceutical application will result in the tailoring of acoustic radiation forces to dramatically enhance such applications.

The waveform to be transmitted is updated and refined after a subsequent transmission of at least one modified waveform pulse by software learning methods performed on received modified waveform pulses at the receivers.

In some embodiments of the invention, the method for controlling mass movement of fluid material within a field of interest comprises:
(a) transmitting at least one original signal of sound waves transmitted from at least one array of transceivers located outside of a field of interest, or from at least one transceiver located inside a field of interest;
(b) receiving the at least one original signal with at least one receiver or transceiver within the field of interest as a received signal; or receiving the at least one original signal in parallel with multiple transceivers located outside the field of interest if the said original signal is transmitted from within the field of interest;
(c) a processor processing the at least one received signal by acoustic time-reversal geometric mean interpolation methods to generate a signal content relationship between the original signal and the at least one received signal;
(d) using the generated signal content relationship to design a modified acoustic waveform that will produce a specific acoustic effect in the field of interest near the at least one receiver; and
(e) transmitting the modified acoustic waveform from the at least one array of transmitters outside of the field of interest to modify mass movement of materials within the field of interest.

In some embodiments of the invention, the ultrasound system for controlling the flow of material injected into a region of interest of tissue comprises:
(a) an array of at least one repositionable array of at least three ultrasound transmitters located outside of the region of interest of the tissue,
(b) output control for the at least three transmitters causing pulse trains to be transmitted from each transmitter in the array,
(c) at least one receiver receiving the pulse train within the region of interest; and
(d) a processor executing geometric mean interpolation software contained in the processor of a computational scheme that computes mass flow based on a transmitted waveform and known characteristics, comprising: at least some existing database of general characteristics within the region of interest or specific patient characteristics derived from radiological imaging within the region of interest to simulate a received wave train at a designated point in tissue in the region of interest, and the processor can iterate until a transmitted waveform results in a designed mass flow, which designed waveform would then be transmitted by the at least three transmitters.

An ultrasound device and software for controlling the flow of material injected into a region of interest of tissue may have and or at least:
(i) an array of at least one repositionable ultrasound array of transmitters located outside of the region of interest of the tissue,
(ii) output control for the phased transmitters causing pulse trains to be transmitted from the transmitters in the array,
(iii) at least one receiver/transmitter receiving the pulse train within the region of interest;
(iv) in some implementations, at least two internal receiver/transmitter transducers to deliver two pulse signals (one from each of the two) to the external array(s) of transducers in order to establish the causal connection between the internal and external transducers as quickly as possible;
(v) a processor capable of executing software for the design of time reversed waveforms for subsequent transmission from the external array, as described above; or
(vi) a processor capable of executing software for computational schemes that compute mass flow based on known characteristics of skull and brain tissue, meninges and blood vessels, to simulate the received wave train at a designated point in tissue in the region of interest.

In the device, an individual patient's image anatomy from three-dimensional radiological imaging may be stored with an information communication link provided from the stored image anatomy to a computer containing a computer algorithm that segments the image anatomy into regions with different acoustical and elastic properties, the software then assigning such acoustical and elastic properties from open database literature, or an individual patient's image anatomy from three-dimensional radiological imaging is stored with an information communication link provided from the stored image anatomy to a computer containing a computer algorithm that segments the image anatomy into regions with different acoustical and elastic properties, the software then assigning each voxel of a brain and skull assigned acoustical and elastic properties derived from said imaging.

The time-reversal approach does assume that the medium (organ in the present context) does not change significantly during the time from the collection of the temporal Green's function data to the time at which the time-reversed signals are sent back into the medium. In a situation where drug perfusion is being performed, it will probably be important to reset (redo) the temporal Green's functions periodically, as they may get stale (that is to say the medium may have changed enough that they need to be updated).

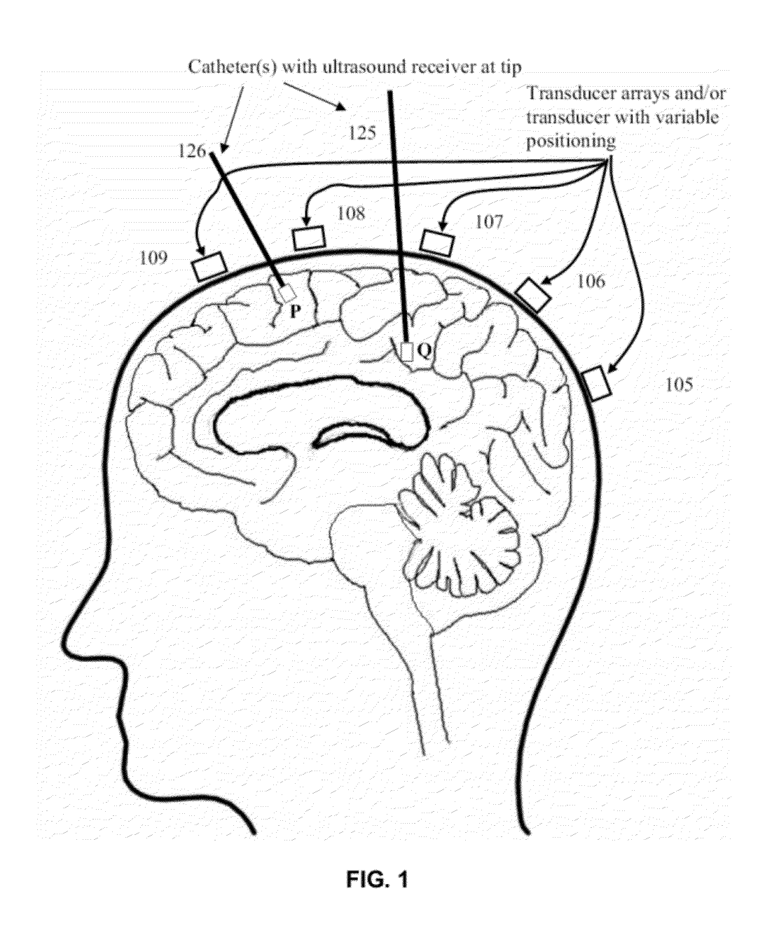

FIG. 1 shows some embodiments of the invention. The ultrasound receivers are within brain parenchyma (as illustrated) and are each contained within a multiple lumen catheter, at least one other lumen being used for infusion of drugs in solution. (The term “drug” will include macromolecules, viruses, cells, and nanoparticles. In general, the active particles that are infused will be such that they cannot effectively cross the blood-brain barrier without the presence of a catheter.) Ultrasound transmitters are placed on the outside of the skull. The figure shows several embodiments within one diagram: (i) The catheters may be just one (e.g. 125) or (ii) multiple (e.g. 125, 126, and possibly others not shown). (iii) The transmitters may be single (e.g. 105) or (iv) multiple (e.g. 105, 106, . . . 109, and possibly others not shown); (v) in addition, a single transmitter is equipped with ancillary frames that allow it to be moved to multiple positions on the skull, such as the positions shown as 106, . . . 109 and possibly others.

The simplest example for application with one catheter infusion port, one receiver and one transmitter, is to design a waveform to prevent backflow (a ubiquitous phenomenon in catheter-based infusions). The backflow phenomenon, which is well known, entails a very thin layer of fluid between the outside wall of catheter and the tissue, which flows up the catheter to a characteristic distance depending on the hydraulic resistance of the tissue, the elastic properties of the tissue, the outside diameter of the catheter, the viscosity of the fluid, and some other parameters. We envision a series of pressure pulses traveling down the (outside) wall of the catheter toward the distal end or tip from which the fluid leaves the catheter to enter the tissue. In order to provide such a series of pulses, one inserts the catheter a short distance into the tissue, sends a pulse through the transmitter, records the pulses at the receiver in the catheter, then advances the catheter an incremental amount, repeats the mentioned process, and so on. Thus one obtains the recorded train of sound at various distances along the track of the final position of the catheter. Then we follow the methods of time-reversal acoustics and send the time reversed trains through the transmitter. This process results in the original pulses (to a good approximation) traveling down the shaft of the catheter as desired to effect retardation of the backflow up the shaft of the catheter.

One implementation of time-reversal methods is included in the following discussion: For the moment, it will be assumed that the catheter material is sufficiently stiff that it can be rotated after emplacement. This assumption is made for clarity of the conception only and is not necessarily recommended or required. Although one could construct a catheter with a second internal cylinder in close contact with the outer cylinder and which could be made to rotate, one approach would be to extend the cylinder from a proximal shielding cannula, image, retract, rotate, extend, and image again as desired. It may be further assumed that at least one transducer (or possibly two, being on the diagonally opposite sides of the catheter opening) is (are) near the end of the catheter. Then, this (these) transducer(s) may be used to localize one (or two) point(s) on the sides of the catheter acoustically. By then ‘rotating’ the catheter in place and mapping out a set of points along a circular arc just outside the catheter may be effected. With one (two diagonally opposite) transducer(s), this acoustic mapping may be done using a 360 (180) degree rotation of the catheter. The rotation would be done discretely, for example in 8 (or 4 respectively) 45 degree turns. At the conclusion of each small turn, an acoustic signal (ping) will be sent from an active transducer at the catheter if available, or from the external active arrays of transducers (if the transducers at the catheter are not active) that should always be available in the methods being described. This collection of pings and the recorded signals associated with each ping measure/establish the Green's functions between the external arrays and transducer(s) at the catheter walls in each rotated location. Then, focusing back on these locations is performed in the standard way (time-reversal focusing) described elsewhere herein for such focusing. It is particularly desirable to provide a minimum of four points (i.e., four 90 degree turns of the catheter if there is a single transducer, or two 90 degree turns if there are two diagonally opposite transducers) around the catheter walls to do the acoustic geometric mean interpolation (also described elsewhere herein) that will produce a ring of higher pressure (acoustic barrier) to discourage backflow of serum along the catheter's external walls. More turns could be used (i.e., eight 45 degree turns), but are not required to produce useful results. Great precision in the catheter rotating process is also not required since all points along the exterior of the catheter are useful points for acoustic barrier focusing. The acoustic geometric mean interpolation process itself also does not require high precision either in that knowledge of the precise locations of the transducers is not needed in the signal processing. It is understood that this invention may be used for backflow prevention without the multiple rotation and replacement of the cannulae, since the catheter diameters are small.

### Array Processing

The effect described above can be obtained, in fact in a superior way, by multiple arrays of transducers since the timing of these can be more precisely controlled (with greater constructive interference at the desired locations thus producing a larger set of controllable effects) than that of transmission of the time-reversed waveforms through a single array of transmitters.

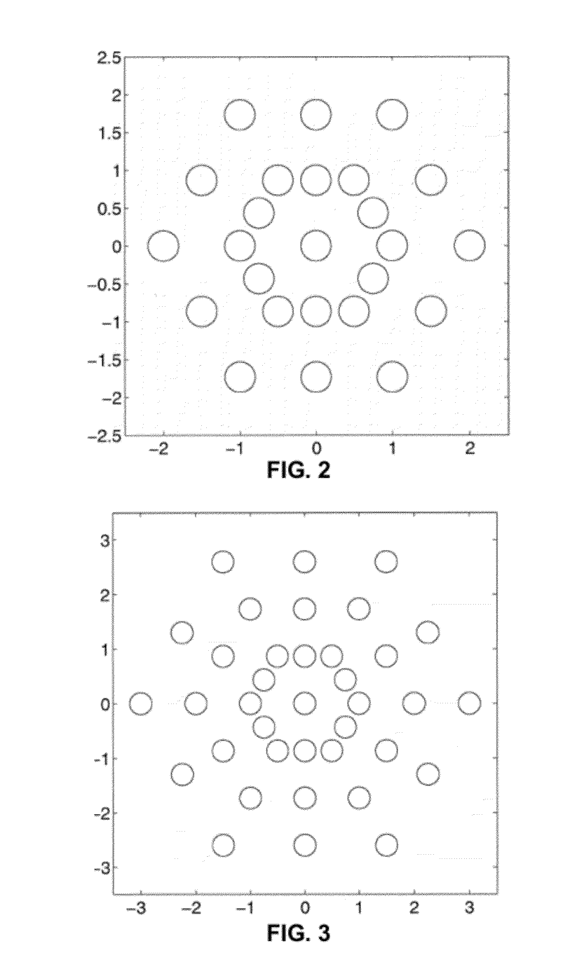

FIGS. 2 and 3 show possible configurations of transceivers within a cluster. FIG. 2 has a hexagonally arranged array with 25 transducers in a cluster while FIG. 3 shows 37 transducers. These arrangements allow for 24 and 36 triples of transducers along a line, respectively. These triples allow for measurement of the curvature of the incoming waves for the purposes of beamforming from time reversal. In the usual embodiment, Green's function can be reconstructed from the intraparenchymal devices being receiver-only. In this case, recordings must be made from each of the external transmitters one by one. Owing to the advantage of parallel reconstruction of Green's functions, another embodiment which can be investigated is that of having transmitters in the intraparenchymal catheters. These transmitters are for the purpose of allowing simultaneous reception by the external transceivers for parallel reconstruction of Green's functions.

Thus, both implementations have at least two catheters in the subject organ (brain, prostate, kidney, or other), both of which can receive and record acoustic signals (passive mode), and in the second case also send acoustic signals (active mode). The two catheters should be in the close vicinity of the nodule (or other object or larger region) to which drug delivery is desired. In the simplest and most rapidly implemented case, the catheter transducers are both active. First, at least one catheter transducer (or one catheter transducer at a time) emits a ping, which is a short burst of sound; then, the at least one second catheter transducer does the same after a period of time (in some cases, a measured or predetermined period of time) has elapsed so the wave train generated by the first ping has dissipated sufficiently. The ping itself has a specific design that is Gaussian in the frequency domain over a typical range of frequencies such as 1 MHz to 30 MHz with a center frequency of 15 MHz (for example), and, as is common in the art, a Gaussian shape that does not fall to zero too rapidly over this frequency range so the resulting pulse shape as observed in the time domain is still well-enough localized in time for the particular application. The resulting signals are recorded at all the transducers (being used in passive mode) in the external array. These recorded signals will be referred to as the effective time-domain Green's functions between the sources and receivers. If the externally recorded signals are then time-reversed (first in last out, last in first out) and simultaneously rebroadcast from the external transducer array elements, a strong and well-defined (though usually not perfectly focused) pulse will then appear at the catheter transducers when this procedure is repeated once for each active catheter transducer. It is desirable that the transducers at both catheters use practically the same pulse shape for their individual pings, and also approximately the same amplitude (loudness) for these acoustic signals. Another implementation of the same ideas can be accomplished using catheters having only passive transducers. This approach takes somewhat longer to implement since the temporal Green's functions must then be measured one at a time between the external active/passive array of transducers and the two passive catheter transducers. The time difference is the difference of a factor of 2 for case of active catheter transducers, and a factor of 2 times the total number of transducers in the external array for the case of passive catheter transducers. Since larger arrays will produce more accurate focusing at the spots of interest, the difference will typically be on the order of 100 times slower. Once these temporal Green's functions are known, however, these two methods become exactly the same. So the differences are only in the data collection time, not in the implementation of the wave interpolation and refocusing, which is just the same in the two approaches.

Another embodiment of this process may be achieved by the generation of microbubbles in tissue: this is described below in the description of FIG. 11.

### Sound Wave Interpolation

Returning to FIG. 1, we now describe further embodiments of the invention. We envisage the placement of other cannulae (e.g., 125 which is not necessarily an infusion catheter as well) into tissue: these must have receivers for the same purposes as outlined above. With the placement of a second cannula, we can now, by geometric mean interpolation, construct a protocol to deliver a specific waveform along the line connecting the two receivers. Equally, we can envisage the placement of up to four transceivers in tissue as being on a sphere, and require a geometric mean interpolation of the response functions received on this sphere. Thus by recording the waveforms received at the respective positions P and Q upon transmission of a pulse from one of the transducers, one can design, by the known principles of time reversal acoustics, waveforms to induce a pulse anywhere along the line PQ or on the arc drawn on a surface such as a sphere. Of course the accuracy of the spatial position of this pulse, as well as its time duration, depend to some extent on the lack of homogeneity of the tissue everywhere, and so will not be perfect. Nevertheless the geometric mean interpolation gives us considerable power for designing such waveforms.

Placement of yet another receiver in a cannula at a third position not in the plane containing the two already placed lines 125 and 126 then allows designed geometric mean interpolations to focus pulses (or indeed any other designed waveforms) anywhere in the parenchymal space, particularly within the triangle defined by the three receivers (approximated as points). Geometric mean interpolations in planar, or spherical, or other regions will allow a variety of applications beyond the prevention of backflow mentioned above. Such applications include enhancing convection of fluid by acoustic streaming along given pathways in parenchymal space. Of course, application of a streaming force in a particular direction (by control of the waveforms) will not always guarantee flow in that direction since the hydraulic resistance of the tissue as well as the anisotropy of such resistance (which is a tensor in general) will determine the actual flow, but flow can be restricted, enhanced or otherwise modified according to the techniques described herein to benefit the delivery or removal of materials within fluid streams in a patient. In other words, the flow direction is the result of the combined interaction of the hydraulic conductivity tensor and the streaming force as a linear motive force. Nevertheless it is clear that designing the streaming force to have certain directions and amplitudes will enhance the controllability of the flow of liquids carrying drugs or other materials within tissue. If the hydraulic resistance were isotropic (not dependent on direction) then the flow would indeed follow the motive force.

When using an arithmetic, geometric, or other interpolation step produces a focused spot of sound at a point on an arc between the at least two catheter transducers. A variety of methods is available for interpolation to those skilled in the art, but the present invention is limited to the geometric mean interpolation method(s) disclosed herein. Thus the most common alternative methods are linear and other, e.g., spline interpolations in multidimensions. Also, if statistical characteristics of the received waveforms are assumed (parametric statistics) or inferred from data (e.g., by kernel estimators), then many methods of statistical interpolation are available. These methods stem from the earliest use of statistics in signal processing, initially for time varying signals. Various cost functions may be used, such as least squares, or inverse-distance weighted least squares, resulting in different signal processing algorithms. Least-squares estimators are popular since elementary calculus can be used to derive the form of the estimator. Both non-parametric methods (such as Empirical Risk Minimization) as well as parametric methods may be used. Also, as those practiced in the art are aware, one can choose other figures of merit, such as the maximum error (to within a tolerance) which may result in better stability of the interpolation, but at the cost of having to obtain a linear programming solution to find the desired estimator. Similarly, other methods may demand quadratic programming. Another use of least squares techniques includes best estimators for lognormal statistics. In the simplest such case, a linear weighting is applied to the logarithms of the Fourier transforms of the waveforms. For two receivers, this is nothing but taking geometric means, and we describe this in more detail below.

It is understood that the analysis presented below is for illustrative purposes, and those skilled in the art will be able to undertake extensions to the more complex cases needed for applications, and to substitute other interpolation for the geometric mean interpolation schemes including those based on statistical estimation and interpolation methods. Such methods, as briefly indicated, include interpolation on spherical surfaces, and other geometric objects.

Geometric interpolation may be accomplished by combining the two sets of recorded signals in the following way: First, a Fourier transform of each of the signals from the pairs already recorded (one from each catheter) at each transducer in the external transducer array is performed. These Fourier transformed signals are combined into a single Fourier transform for each external transducer by multiplication using a geometric mean. Then, the resulting Fourier transformed signals are inverse transformed back into the time domain. The resulting time-domain signals are then treated just like the originally recorded signals, time-reversed and re-emitted back into the medium/organ. By varying the exponent x from zero to unity, the focal spot can be moved from one of the catheter locations to the other one, gradually, in steps. For example, a sequence of moves typified by x=0.0, 0.05, 0.1, 0.15, etc., would move the sound spot in steps of approximately 5% of the distance between the two catheters with each refocusing event. The step sizes will need to be coordinated with the size of the spot itself, and this will also depend on the choice made concerning the Gaussian temporal size of the original acoustic pings from the two catheters.

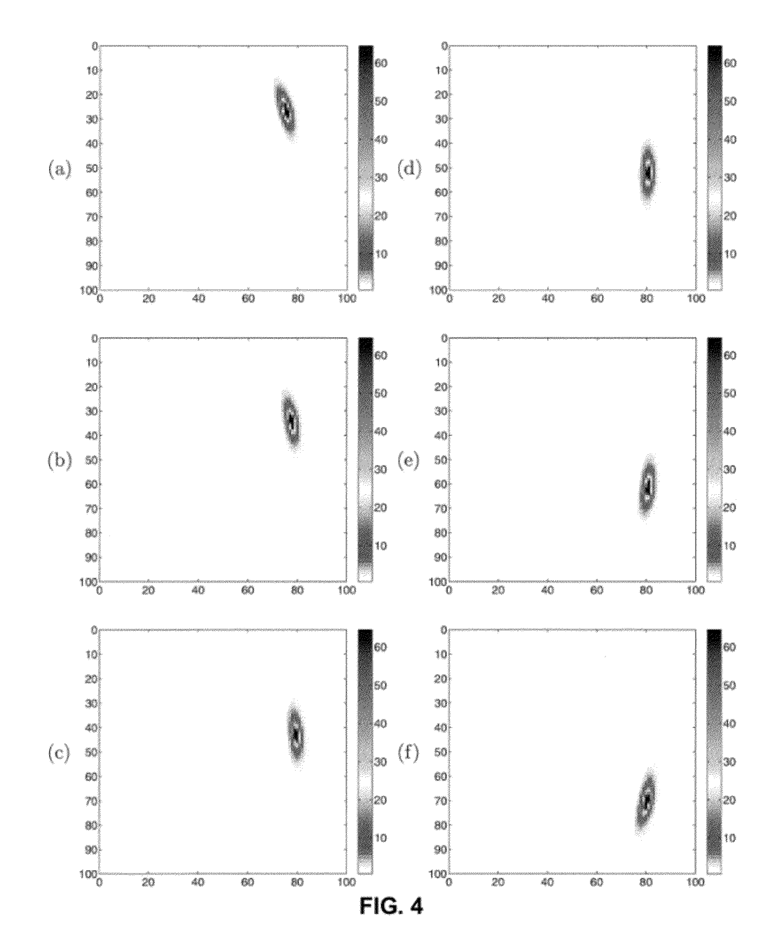

FIG. 4 shows an example of sound wave interpolation. The acoustic array is located along the left hand side of each figure, and consists of 21 ideal point transducers equally spaced from vertical positions 20 to 80. Distance along each side of these square figures is 15 cm. The two interior transducers are located at points (75.025, 25.025) and (80.025, 70.025). The center frequency is 5 Mhz. Nominal wave speed is 1.5 km/sec. Step sizes in units of the distance between the internal transducer locations are respectively (a) x=0.01, (b) x=0.20, (c) x=0.40, (d) x=0.60, (e) x=0.80, and (f) x=0.99. The centers of the interpolated acoustic spots do not line up along a straight line between the end points, but rather along an arc that bows slightly away from the external array location.

### Geometric Mean Interpolation for Waves in Homogeneous Media

For purely illustrative purposes, we discuss the behavior of geometric mean interpolators, for waves in homogeneous media. It is assumed that signals from the at least two emplaced sound sources have some known pulse shape in the frequency domain, which we will label s(ω,\vec{r}ᵢ), where i=1, 2 refer to the two sound sources, and rᵢ are their locations in three dimensional space. The angular frequency is ω=2πf, where f is the frequency in Hz. For our present purposes, it will turn out that the precise shape of the wave pulse s is not critical, but it is important that the same shape be used at the two emplaced sound sources, so s(ω,\vec{r}ᵢ)=s(ω,\vec{r}₂). One methodology is using a Gaussian pulse shape in the frequency domain, but one with not too narrow of a frequency band, as it is also important to have a finite temporal duration signal from our two sources. The resulting signal received at one of the transducers in the external array is of the form:

Ψᵢ(\vec{r},t)=∫G₀(ω;\vec{r},\vec{R}ᵢ)s(ω,\vec{R}ᵢ)exp(iωt)dω/\sqrt{2π}.

The acoustic Green's function for a homogeneous isotropic medium in three dimensions is:

G₀(ω;\vec{r},\vec{r}ᵢ)=expik|\vec{r}−\vec{R}ᵢ|/4π|\vec{r}−\vec{R}ᵢ| for i=1,2.

It may be assumed that the inverse Fourier transform has been performed, thus giving:

$${\int }_{0}^{∞} exp ( - iω t ) {Ψ}_{i} ( {r}^{->} , t ) \frac{d t}{\sqrt{2 π}} = {G}_{0} ( ω ; {r}^{->} , {{R}^{->}}_{i} ) s ( ω , {{R}^{->}}_{i} )$$

$$for i = 1 , 2.$$

It may be also assumed that such data is collected in this way over a set of acoustic array elements at spatial locations \vec{r}=\vec{r}ₙ, where n=1, . . . , N and is typically on the order of 50 or 100.

To produce spots of soundwave concentration at particular points in the region between the two points \vec{R}₁ and \vec{R}₂, the data must combined in some way. The procedure of doing this will be called “soundwave interpolation.” One way of accomplishing this is to combine these data using a geometric mean of the received waveforms. It is easily seen that this choice of signal design removes the pulse shape from our problem, and we only need to analyze the resulting form

Gₓ(ω;\vec{r},\vec{R}ₓ)≡[G₀(ω;\vec{r},\vec{R}₁)]^{x}[G₀(ω;\vec{r},\vec{R}₂)]^{y},

where x+y=1, x and y being real numbers in the range [0,1], and \vec{R}ₓ=x\vec{R}₁+y\vec{R}₂ is a point along the line connecting the end points \vec{R}₁ and \vec{R}₂. The numbers x and y are relative weights, and for the special case

$$x = y = \frac{1}{2}$$

the value \vec{R}ₓ is exactly the mid-point of this connecting line. It can be shown that the vector argument \vec{R}ₓ, while providing the nominal location of the refocused (interpolated) spot, is not the true spot location. Instead the true interpolated spot actually appears along an arc that is slightly deviated from the spatial locations mapped out by the straight line \vec{R}ₓ (as x varies from 0 to 1) in most cases.

Analysis shows that this scheme essentially equivalent to focusing our spot at a point that is the distance \sqrt{x|\vec{r}−\vec{R}₁|²+y|\vec{r}−\vec{R}₂|²} away from one of the elements of the array. This analysis depends on the assumption that the distance between the two spots within the tissue of interest where the transducers attached to the catheter are placed is small compared to the distance of either of the transducers to any of the transceivers in the external array. All the array elements are acting simultaneously and the final result will be constructive interference in the vicinity of the location \vec{R}ₓ, but slightly beyond this point. So a series of focusing events using an array of transducers will trace out an arc that moves from \vec{R}₂ to \vec{R}₁ as x varies from 0 to unity. The focal spots will not be exactly along the straight line between \vec{R}₂ and \vec{R}₁, but instead fall along a well-defined arc connecting the two points.

The final step in the processing takes the Fourier transform of the modified (averaged) Green's functions for each transducer in the array and rebroadcasts the time-reversed signal, just as in normal time-reversal processing. The signals that are broadcast are the time-reversed versions of

$${\overline{Ψ}}_{x} ( {r}^{->} , t ) = \int {G}_{x} ( ω ; {r}^{->} , {{R}^{->}}_{x} ) s ( ω , {{R}^{->}}_{x} ) exp ( iω t ) \frac{d ω}{\sqrt{2 π}} ,$$

simultaneously from each location of an array transducer \vec{r}=\vec{r}_{y}, for n=1, . . . , N.

FIG. 4 shows how the focal spots move as the value of parameter x is changed in the interpolation scheme described above. Therefore, by interpolating the signal data, a virtual point response can be determined by the weighting of signals from the at least two different sources. This point response can be used to determine and provide a signal from the multiple sources that will have a designed and desired effect on a specific location within a patient or land mass.

Thus, it has been shown herein that soundwave interpolation can be accomplished using a combination of time-reversal data collection, frequency domain data processing involving the geometric mean of the empirical Green's functions, and subsequent rebroadcasting of the signals from the external array in the reversed-time domain. Caveats that should always be kept in mind include: if the medium is heterogeneous in space (the usual case), the rebroadcast signal may be only weakly received at the original location. The focus at this location may be either better or worse than normally expected (when measuring against the Rayleigh criterion), since spatial heterogeneity can actually improve the focus—due to the presence of greater angular diversity in the received signals, while simultaneously creating extra sources of amplitude loss. Temporal changes in the medium will naturally degrade and eventually invalidate this procedure over longer time periods, since the up-to-date Green's function of the medium may not be the same as the one at the time the original (eventually stale) signal recordings were made. Thus, in environments like the ocean, it is normal to observe gradual decline in the ability to focus using stale (older) Green's function data. For the main types of applications envisioned by the inventors (to biomedical problems such as drug perfusion problems in brain, prostate, or other tissue and organs), there is no anticipation from the teachings in this art that temporal degradation would be a major source of difficulty with the application of the methods outlined herein.

One way of characterizing this method is as a method of providing a focused soundwave using soundwave interpolation procedures comprising: providing signals from at least two positioned sound sources having known positions in three-dimensional space and known pulse shape in a frequency domain; receiving signals at a transceiver within a target volume; producing spots of soundwave concentration at particular points in the target volume along a line or an arc between at least two identified points within the volume; combining data related to received signals by a geometric or arithmetic means relatively weighting the data at or between the at least two points, and determining at least one weighted value for a signal from the at least two positioned sound sources.

The Weighted Value Removes Significant Factors of Pulse Shape from the Received Signals

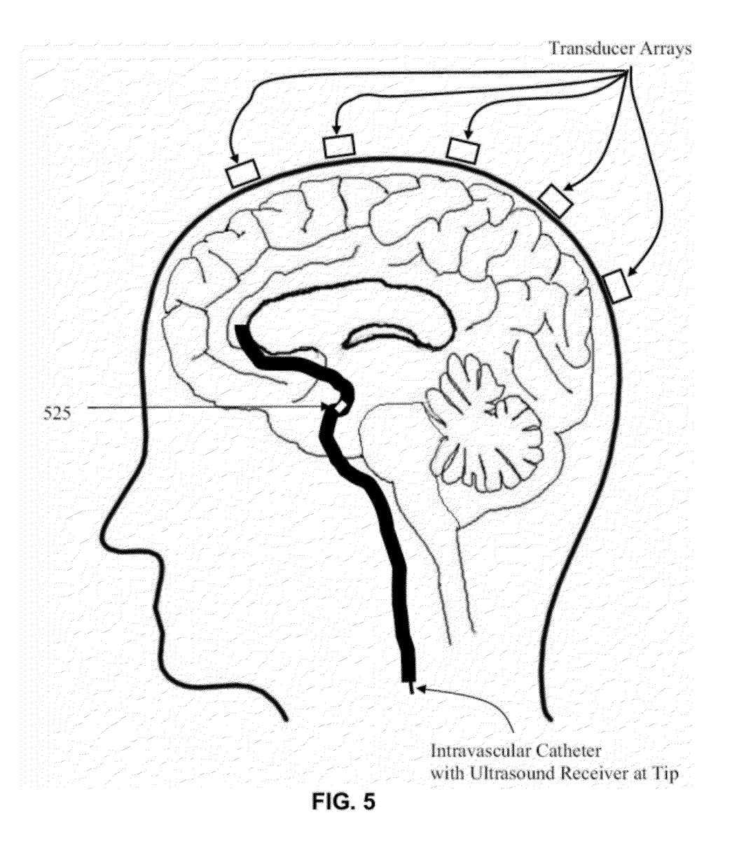

FIG. 5 shows an endovascular embodiment of technology within the scope of the generic invention. The catheter, which may be a microcatheter, has one or more receivers along its length. It is inserted into blood vessels in the brain. In the case of one receiver at its distal end (425), such a receiver must be positioned as close to the region of brain parenchyma where the drug is to be administered. In the case of two receivers, an aim would be to deliver this therapy to a region of the brain distributed radially around the line (or proximate to the line) joining the two receivers as an axis. The therapy is then delivered endovascularly, such as by a catheter or by injection, and the invention is used to focus ultrasound in the region of the vessel at the receiver or between the receivers. The sonoporation that results from the focusing of the sound is expected to increase the blood brain barrier permeability and allow increased efflux of drug into brain parenchyma for therapeutic effectiveness. An advantage of this invention is that transmitter technology and electronics need not be inserted into the blood vessels, resulting in a significant advantage in cost in the single-use catheter, as well as reducing the requirements for safety due to the significant reduction in power in the electronics that is inserted into the body. It may be envisaged further increasing the effectiveness of the sonication by providing streaming or sonic guidance to distribute the therapeutic particles further into the tissue. In addition, the level of sonoporation (power, frequency, etc.) may be optimized by incorporating the known pharmacokinetics of the therapeutic molecule so that a desired level of penetration into tissue may be effected.

It is understood that the receiver need not be inserted into the blood vessels, but may be placed in tissue in a region of the brain targeted for perfusion by the drug, so that blood vessels nearby may be sonoporated by this technique. A particular embodiment of this invention is in conjunction with microcatheters introduced into the venous system close to almost any desired position in brain parenchyma. The vein there may then be punctured, and a systemically administered agent would then have privileged access to that region of parenchyma adjacent to the punctured vein. Ultrasound focus could be provided by the afore-described time reversal techniques to distribute the agent favorably in the brain.

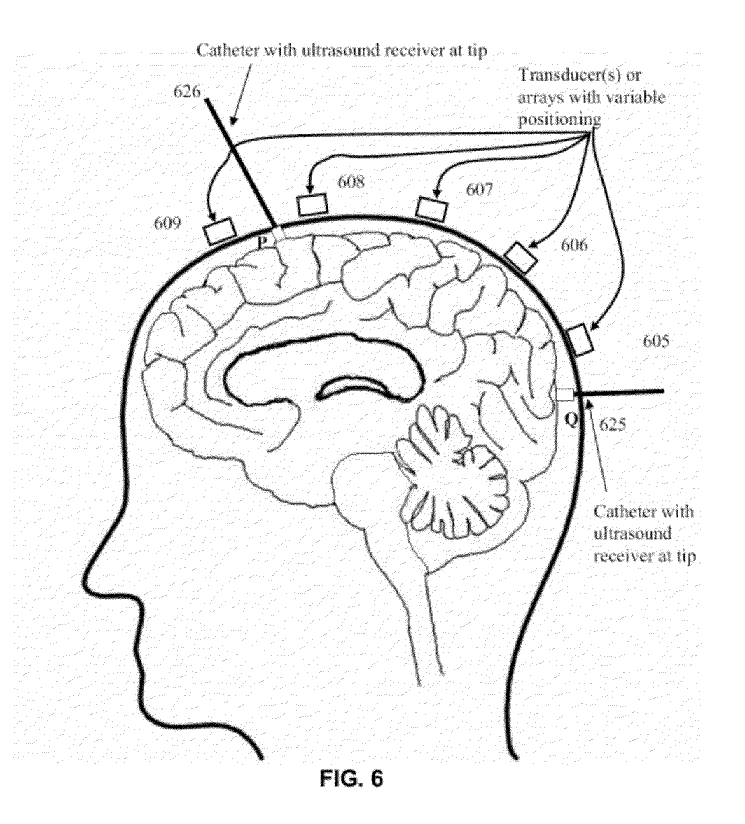

FIG. 6 shows an application of the invention within the generic scope of the invention to intrathecal delivery. Here, the invention performs two separate functions. The intent is to get drug or therapeutic particles into cortical tissue. The cortex is enveloped in a thin layer of cells comprising the pia mater. The cerebrospinal fluid (CSF) flows in the sub-arachnoid space between the pia and the dura mater. Intrathecal delivery involves injection of therapy into the CSF, usually in the spinal column, though it can also be performed sub-durally. However, such a procedure would be very unlikely to deliver the drug into the cortex for two reasons: first the flow of the CSF will more likely move the therapy away from the intended cortical targets to the CSF drainage areas, and secondly, the pia itself is a barrier to the entry of especially the larger molecules, as the BBB is (though the pial junctions are not as tight). One application of the present invention may be aimed at overcoming both these difficulties. A focus of the ultrasound will be near the site of the intrathecal delivery (625). A phased delivery of ultrasound will direct the flow of the therapy to near the site of the intended cortical penetration (626). A different beam formation will then attempt sonoporation to open the pial barrier reversibly to allow and direct entry into the cortical matter. The methods of beam forming and of time reversal to focus and phase the ultrasound will be as described before.

The method can also be practiced as an application to oil industry problems, where a common issue is that certain oil reservoirs have known fluids that are stuck underground, and it would be helpful to shake them up a bit and get them moving again. Similarly in the environmental community where certain contaminants are present underground, it would be advantageous to be able to shake certain contaminants loose and control the direction in which they flow so they do flow to the desired contaminant collection point.

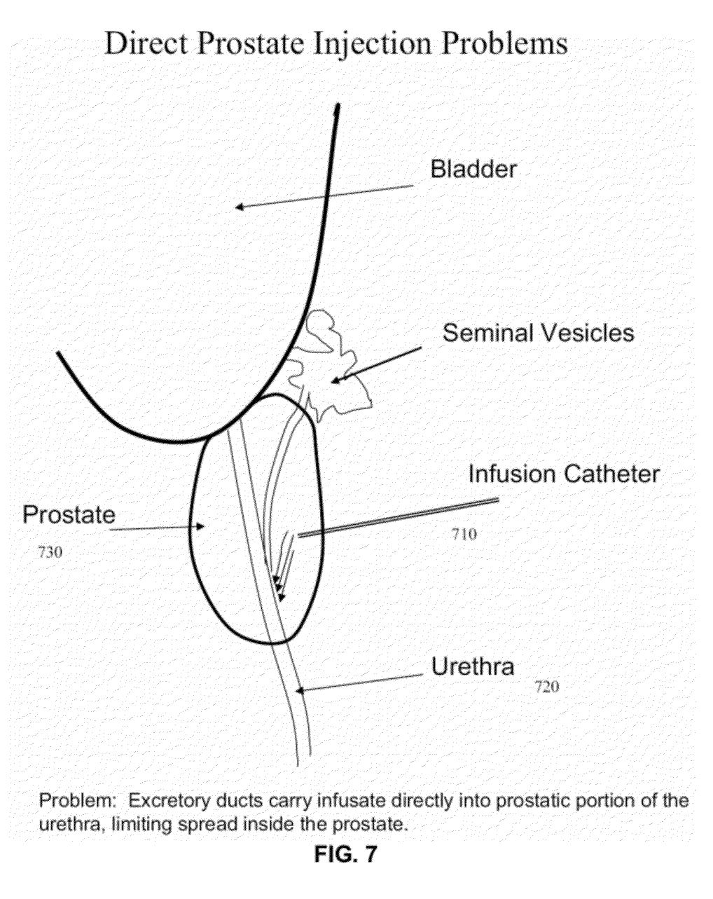

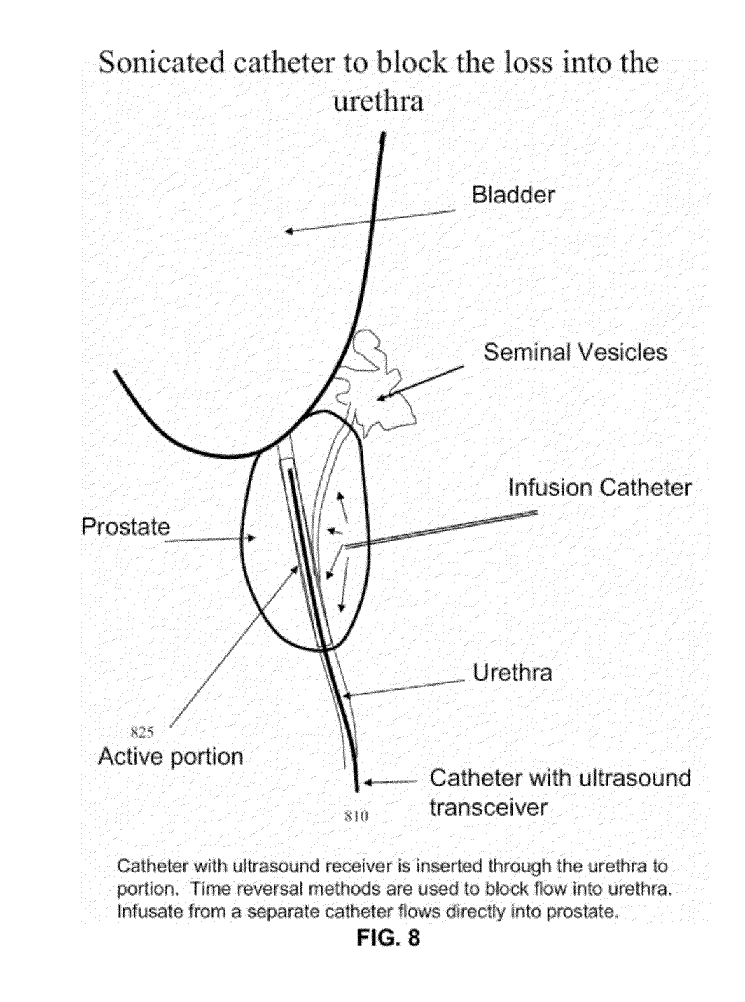

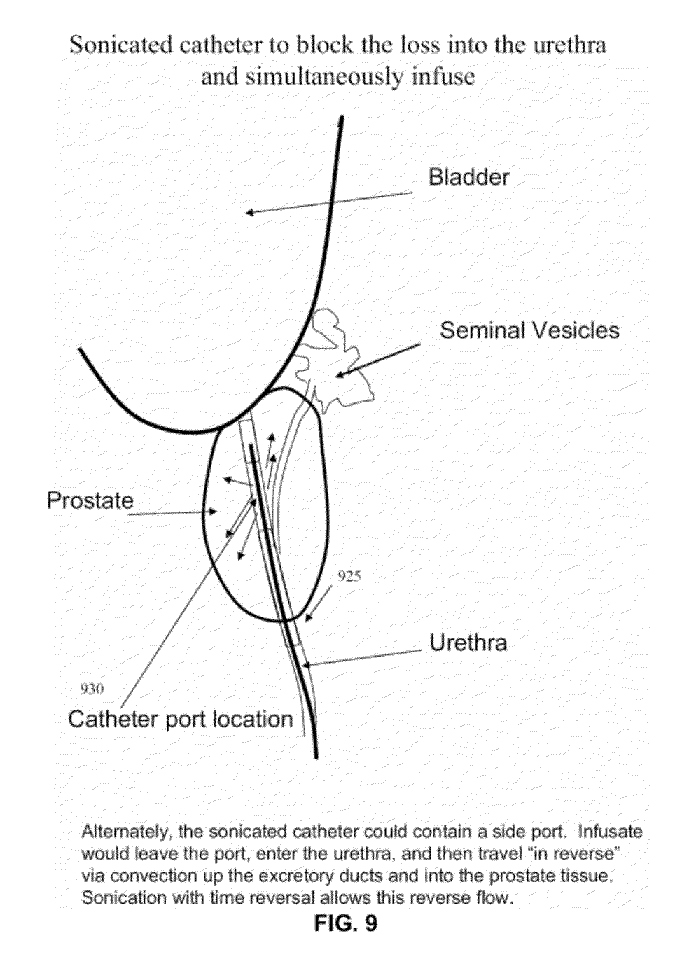

FIGS. 7-9 show schematic representations that envisage an embodiment of the invention for prostate therapy wherein fluids are infused intraparenchymally into the prostate. As shown in FIG. 7, when drug is infused from one or several injection sites within the prostate, (only one of which is shown in 710) a problem to avoid is that the fluid flows into ducts which lead into the prostatic urethra 720. The fluid-filled ducts are a low pressure sink for fluid flow and this makes it difficult to obtain a uniform distribution of the infusate or the drug carried therein. One embodiment of the present generic technology of the present invention is to introduce a catheter with ultrasound transceiver, or two transceivers, through the urethra into the prostate. In particular embodiments, the monitoring of this process by MR imaging or other means is preferred. The waveform desired at the location of the receiver within the volume of prostatic tissue is then fed into the transmitter array which is located outside. The methods of time reversal focusing as outlined above are then used to direct a streaming force away from the prostatic urethra into the tissue. This will force the infused drug away from the urethra and allow it to distribute more evenly within the prostate.

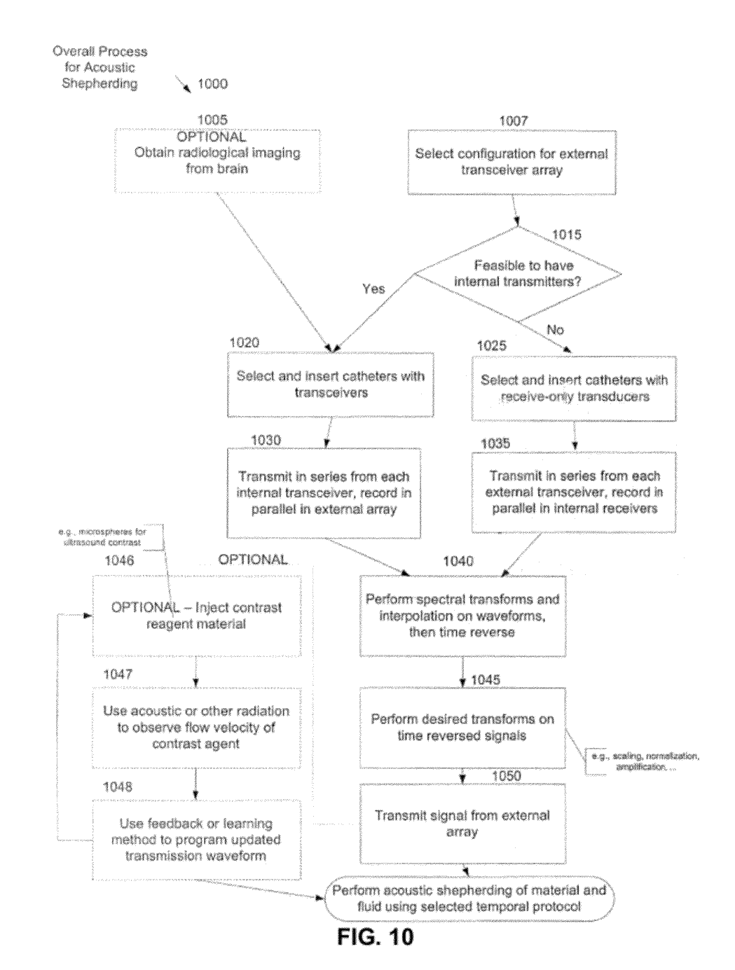

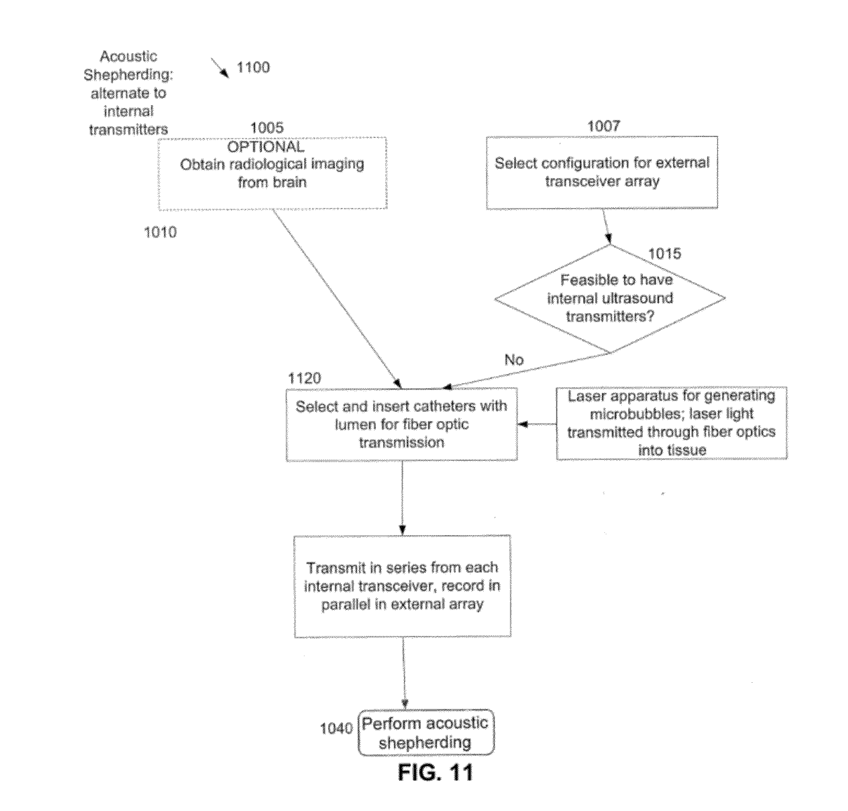

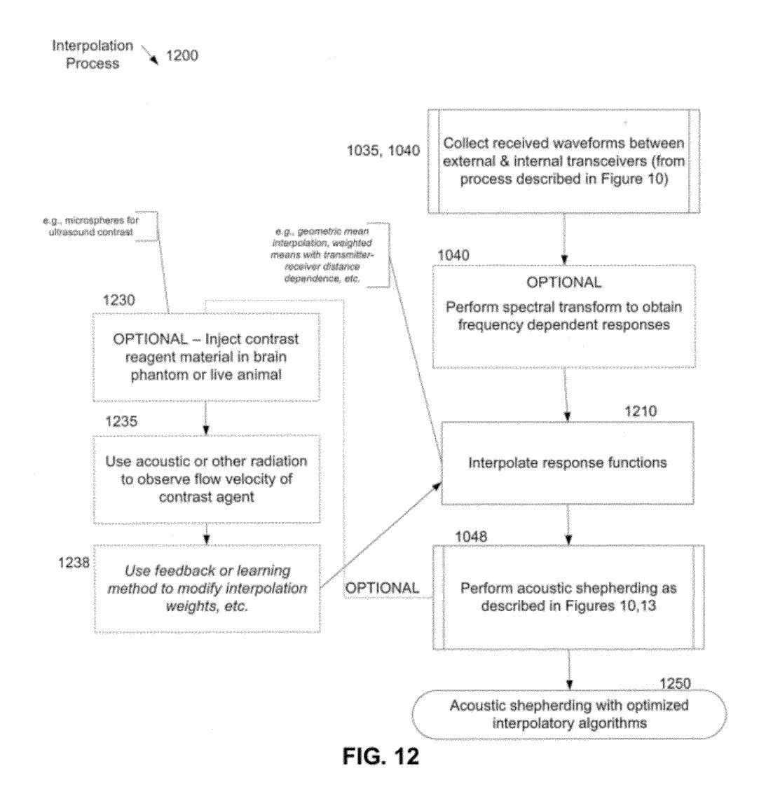

FIG. 10 shows a flow chart of a specific process within the generic concepts according to the present invention. There is a first optional step 1005 in which a radiological (or other format) image is taken of the general locus of the planned treatment, here indicated as the brain. To assure a beneficial image 1007, the user selects or designs a configuration for the external transceiver array to be used in transmitting sonogram pulses and signals into the area of interest of the patient. In step 1015, it is determined whether it is feasible to use internal transmitters in providing the signal. One skilled in the art would be aware of the basis of selection of such parameters, such as ease of access, potential for damage from insertion, removal or proximal signals, and the like. If the answer is yes, in next step 1020 one selects and inserts catheters with transceivers into the appropriate area of the patient. A next step would include transmitting, most likely in series, but in a designed pattern is also possible, pulses or transmissions from each internal transceiver. The signals are then recorded in parallel in the external array. At this point, after using internal transceivers, spectral transforms and interpolations on waveforms would be performed and time reversed 1040.

If the answer in step 1015 was no, then the procedure would move to alternative step 1025 where one selects and inserts catheters with transceivers into the appropriate area of the patient. A next step would include transmitting, most likely in series, but in a designed pattern is also possible, pulses or transmissions from each internal transceiver. The signals are then recorded in parallel in the external array. At this point after using internal transceivers, spectral transforms and interpolations on waveforms would be performed and time reversed 1040.

In each case, after step 1040, one could then perform in step 1045 desired transforms on the desired time-reversed signals, such as scaling, normalization, amplification and the like. Once the signal data have been provided in a form designed to have the local sonication effect within the patient in the area that has been evaluated, an actual procedure 1050 can be performed where the operator transmits the altered signal towards the target area to have the effect desired from the adjusted or altered signal, such as performing Acoustic Shepherding of material and or fluid.

The method can be improved with the use of contrast agents to permit visualizing the effects of acoustic shepherding. For example, FDA approved microbubbles or microspheres may be co-infused with the therapeutic agent, or at the beginning of the infusion with the therapeutic agent as shown in step 1046. A transmit waveform of a frequency resonant with the microspheres, with time-gated reception at the external array will provide information about the success of the shepherding. Thus the movement of the particles is observed 1047 by acoustic or other radiation, detecting or measuring flow velocity of contrast agent. Feedback 1048 is then used to evaluate, appreciate or analyze and learn about movement of the injected material. Once such feedback is obtained, the method can be augmented with learning methods such as neural network methods, genetic algorithms, least square estimations, density estimators, and support vector machines.

It is understood that microspheres may be too large for several applications such as drug transport through the interstitium of gray matter in the brain. In such cases, nanospheres with sufficient contrast may be used. Gold and other materials inert or non-interacting with live tissue may be used as appropriate.

FIG. 11 illustrates that sound sources inside the brain (e.g., when attached near the tips of the catheters or other placement systems) could either be similar to those in the external transducer array (for example, piezoelectric transducers) or they could be of a different type of external transducer array, making use of optical fibers to send light pulses that will heat the fluids locally and create sound sources in the form of cavitating bubbles in the liquid. Such methods for creating cavitating bubbles can follow the disclosure of U.S. Patent Application Publication 2004/005437, published Mar. 18, 2004, titled “Method and system to create and acoustically manipulate a microbubble” by Matthew O'Donnell; hereby incorporated by reference. The feasibility of this approach for creating microbubbles was demonstrated in the publication “Mapping elasticity in human lenses using bubble-based acoustic radiation force” by Kyle W. Hollman, Matthew O'Donnell and Todd N. Erpelding, published in Experimental Eye Research. Vol. 85, pp 890-893 (2007); hereby incorporated by reference.

FIG. 12 is a flow chart indicating one format of an interpolation process comprising some of the steps described in detail above. In the embodiment shown in FIG. 12, the received waveforms have been collected by the process described in FIG. 10 (or FIG. 11), comprising the set we denote G_{IE}, where I=1, 2, . . . J count the internal transducers, and E=1, 2, . . . N count the external transducers in one or more arrays. These waveforms are collected over a long enough time following a pulsed transmission, so that one is assured that the received signals have decayed to noise-only levels. Then a spectral transform is performed (e.g., by way of a fast Fourier transform—and those skilled in the art may utilize other techniques as appropriate such as wavelet transforms), also as previously described. An interpolation of the received waveforms is performed by arithmetic, geometric, or other interpolation processes (1210). In an optional process (such as in a particular embodiment as animal experimentation), contrast reagents, visible under some form of imaging such as ultrasound or Magnetic Resonance (MRI), may be inserted into the medium (1230) and monitored (1235) to observe the focal effects of the interpolation. The interpolation method, such as the weights used in a geometric or arithmetic mean, may then be altered by a learning method such as a neural network (1238). Such a process could be used to improve the accuracy of the Acoustic Shepherding process.

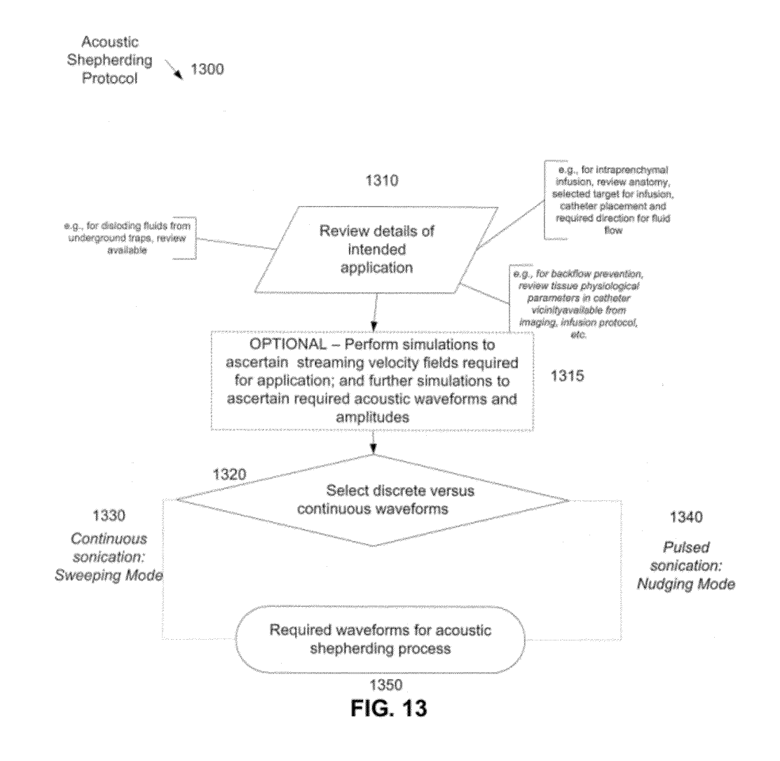

In FIG. 13, it is indicated in the flow diagram that different applications may call for fundamentally different time sequences for the Acoustic Shepherding protocol. Thus have we envisaged in the practice of the present technology that a pulsed mode with periodic or intermittent pulses (termed the Nudging mode) as well as one with continuous transmission (which can be termed the Sweeping mode).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for controlling mass movement of a fluid material within a field of interest comprising:
(a) transmitting at least one original signal of sound waves from at least one array of transceivers located outside of a field of interest, or from at least one transceiver located inside the field of interest;
(b) receiving the at least one original signal with at least one receiver or transceiver within the field of interest as a received signal; or receiving the at least one original signal in parallel with multiple transceivers located outside the field of interest if the said original signal is transmitted from within the field of interest;
(c) processing the at least one received signal by a processor using an acoustic time-reversal geometric mean interpolation method to generate a signal content relationship between the original signal and the at least one received signal;
(d) designing a modified acoustic waveform using the generated signal content relationship to produce a specific acoustic effect in the field of interest near the at least one receiver; and
(e) transmitting the modified acoustic waveform from the at least one array of transmitters outside of the field of interest to modify mass movement of materials within the field of interest.

2. The method of claim 1, further comprising introducing the fluid material into the field of interest.

3. The method of claim 2, wherein said introducing step comprises injecting the fluid material into the field of interest.

4. The method of claim 1, wherein the field of interest is a tissue within a subject.

5. The method of claim 4, wherein the subject is a human subject.

6. The method of claim 1, wherein the fluid material is oil or hazardous waste and the field of interest is underground earth.

7. The method of claim 1, wherein the transmitters are ultrasound transmitters.

8. A system for controlling the flow of a material injected into a region of interest, comprising:
(a) an array of at least one repositionable array of at least three transmitters located outside of the region of interest;
(b) an output control for the at least three transmitters causing pulse trains to be transmitted from each transmitter in the array;
(c) at least one receiver receiving the pulse train within the region of interest; and
(d) a processor executing geometric mean interpolation software contained in the processor of a computational scheme that computes mass flow based on a transmitted waveform and known characteristics, comprising: at least some existing database of general characteristics within the region of interest or specific patient characteristics derived from radiological imaging within the region of interest to simulate a received wave train at a designated point in tissue in the region of interest, and the processor can iterate until a transmitted waveform results in a desired mass flow, which designed waveform would then be transmitted by the at least three transmitters.

9. The system of claim 8, wherein the receiver is a transceiver.

10. The system of claim 8, wherein the transmitters are ultrasound transmitters.

11. The system of claim 8, wherein the field of interest is a tissue within a subject.

12. The method of claim 11, wherein the subject is a human subject.

13. A method for controlling flow of a material introduced into a tissue, comprising:
(a) providing a set of at least one ultrasound transmitter array located outside of the region of interest in the tissue;
(b) providing at least one signal from the at least one ultrasound transmitter, which can be either internal or external to the field of interest and within and/or without a patient, if used in a medical procedure;
(c) providing at least one receiver/transmitter located within the tissue; and
(d) implementing time-reversal acoustics by steps comprising (i) recording the at least one signal from the at least one ultrasound transmitter as a pulse or pulse trains transmitted from the at least one transmitter in the array, (ii) modifying the received waveform in a pre-determined manner to affect an acoustic result within the tissue including geometric mean interpolations, (iii) time-reversing the modified signal to form a time-reversed waveform, (iv) further modifying the time-reversed waveform including scaling, normalization, and amplification in a pre-determined manner to affect an acoustic result within the tissue, and (v) re-transmitting from the transmitter array as a modified waveform pulse.

14. An ultrasound device or system for controlling the flow of material injected into a region of interest within a tissue, comprising:
(a) an array of at least one repositionable array of one or multiple ultrasound transmitters located outside of the region of interest of the tissue,
(b) an output control for the transmitters causing pulse trains to be transmitted from the transmitters in the array,
(c) at least one receiver for receiving the pulse train within the region of interest; and
(d) a processor capable of executing software contained in the processor of a computational scheme of geometric mean interpolation that computes mass flow based on known characteristics of the tissue.
